(19) United States
(12) Patent Application Publication
(10) Pub. No.: US 2007/0054960 A1
(43) Pub. Date: Mar. 8, 2007

(54) Sertraline acid addition salt, its preparation and its use in the preparation of sertraline hydrochloride form II

(76) Inventors: Ramana Venkata Kintali, Thane (West) (IN); Johannes Ludescher, Breitenbach (AT); Raji Nair, Mumbai (IN); Sudhir Sawant, Thane West (IN)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/516,395

(22) Filed: Sep. 6, 2006

(30) Foreign Application Priority Data
Sep. 6, 2005 (GB) 0518135.9

(51) Int. Cl. A61K 31/205; C07C 229/02

(52) U.S. Cl. 514/554; 562/480; 562/573; 562/590

(57) ABSTRACT

The present invention relates to novel acid addition salts of sertraline, their preparation and their use in the preparation of crystalline Form-II of Sertraline hydrochloride.

The present invention relates to novel acid addition salts of sertraline, their preparation and their use in the preparation of crystalline Form-II of Sertraline hydrochloride.

Sertraline hydrochloride chemically known as (1S,4S)-4-(3,4-Dichlorophenyl)-1,2,3,4-tetrahydro-N-methyl-1-naphthalenamine hydrochloride is first disclosed in EP 30081. U.S. Pat. No. 5,248,699 discloses five crystalline forms of sertraline hydrochloride, designated as Form I, Form II, Form III, Form IV and Form V and a process for the preparation thereof. According to U.S. Pat. No. 5,248,699 Form II is obtained by rapid crystallization of sertraline hydrochloride from an organic solvent, including isopropyl alcohol, hexane, ethyl acetate, acetone, methyl isobutyl ketone, glacial acetic acid and ethyl acetate.

WO2004/065348 is directed to pharmaceutically acceptable salts of sertraline and said salts are selected from the group consisting of the p-toluenesulfonic acid salt, the fumaric acid salt, the benzenesulfonic acid salt, the benzoic acid salt, the L-tartaric acid salt and the (−)-camphor-10-sulfonic acid salt and process for the preparation thereof.

WO2005/000786 is directed to an acid salt of sertraline, wherein the acid is citric acid, fumaric acid, malic acid, maleic acid, malonic acid, phosphoric acid, succinic acid, sulfuric acid, L-tartaric acid, HBr, acetic acid, benzoic acid, benzenesulfonic acid, ethanesulfonic acid, lactic acid, methanesulfonic acid or toluenesulfonic acid, a process for the preparation thereof and a pharmaceutical composition comprising an acid salt of sertraline.

WO2005/012224 discloses a method for preparing pure sertraline hydrochloride form II by using sertraline as base or citrate salt.

The present invention relates to new acid addition salts of sertraline wherein the acid is selected from glutamic acid, salicylic acid, adipic acid, phthalic acid, 4-methoxy benzoic acid and 4-methyl benzoic acid.

The present invention further provides a process for preparing the novel sertraline salts by stirring sertraline base in an organic solvent with an organic acid selected from glutamic acid, salicylic acid, adipic acid, phthalic acid, 4-methoxy benzoic acid and 4-methyl benzoic acid at a suitable temperature for about 2 to 6 hours and isolating the formed sertraline salt from the solution by a conventional method.

Preferably sertraline base is dissolved in a solvent selected from methanol, ethanol and/or ethyl acetate. An organic acid which may be dissolved in methanol, ethanol and/or ethyl acetate is added to the solution of sertraline base.

The acid addition salts of sertraline according to the invention are obtained in high yields combined with high purity.

Further the present invention relates to a process for preparing sertraline hydrochloride Form II from an acid addition salt of sertraline wherein the acid is selected from glutamic acid, salicylic acid, adipic acid, phthalic acid, 4-methoxy benzoic acid, 4-methyl benzoic acid and (R)-mandelic acid by slurrying a sertraline salt in an organic solvent or organic solvent mixture at a temperature between ambient temperature and the boiling point of the solvent or solvent mixture, preferably between 25-80° C., followed by treating with the source of hydrochloric acid, stirring the resulting mixture at a temperature between ambient temperature and the boiling point of the solvent or solvent mixture, preferably between about 20 and 80° C. for a period of time sufficient to effect the transformation to sertraline hydrochloride Form II. Preferably the mixture is stirred between 1 and 4 hours. Suitable solvents for slurrying a sertraline salt include ethyl methyl ketone, acetonitrile, methyl isobutyl ketone, nitromethane, nitroethane, methoxypropyl acetate, 2-ethoxy ethanol, methyl formate, ethylformate, isopropyl acetate, 2-ethoxy ethanol, ethylacetate, n-propylacetate, n-butylacetate and/or isobutyl acetate. As used herein, “source of hydrochloric acid” includes gaseous hydrogen chloride, aqueous hydrogen chloride, hydrogen chloride as a solution with an organic solvent such as isopropyl alcohol and diethyl ether, and hydrochloride of an organic amide. Preferred hydrochlorides of organic amides are selected from hydrochlorides of C₁₋₄-aliphatic acid amides, C₁₋₄-aliphatic acid N—C₁₋₂-alkylamides, C₁₋₄-aliphatic acid N,N—C₁₋₂-dialkylamides, a 5 or 6 membered cyclic aliphatic amide and/or a 5 or 6 membered cyclic aliphatic N—C₁₋₂-amide. Most preferred hydrochlorides of organic amides are dimethylacetamide hydrochloride and/or N-methylpyrrolidone hydrochloride. The hydrochloride source may be used in stoichometric amounts or an excess amount between 2 and 5, preferably 2 and 3 mol equivalents compared to the acid addition salt of sertraline.

Sertraline hydrochloride Form II prepared according to the present invention has a high purity and may be used in pharmaceutical compositions together with pharmaceutically acceptable carriers and excipients.

### EXAMPLES

### Example 1

### Preparation of Sertraline Phthalate

To a solution of sertraline base (16.3 g) in ethyl acetate (250 ml) was added a solution of phthalic acid (8.47 grams) in ethanol (55 ml) . The resultant solution was stirred at 25-30° C. temperature for 5 hours. The solvent was distilled off under reduced pressure and the residue obtained triturated with petroleum ether (80 ml). The slurry was filtered, washed with petroleum ether (10 ml) and dried under reduced pressure at 50° C. resulting in 23 g (92%) of sertraline phthalate (melting point: 140.3-142.4° C.).

### Example 2

### Preparation of Sertraline Glutamate

To a solution of sertraline base (14.38 g) in ethyl acetate (300 ml) was added glutamic acid (7.2 g). The resultant solution was stirred at 25-30° C. temperature for 5 hours. The solvent was distilled off under reduced pressure and the residue obtained triturated with petroleum ether (80 ml). The slurry was filtered, washed with petroleum ether (10 ml) and dried under reduced pressure at 50° C. resulting in 14 g (67%) of sertraline glutamate.

### Example 3

### Preparation of Sertraline Salicylate

To a solution of sertraline base (14.38 g) in ethyl acetate (300 ml) was added a solution of salicylic acid (6.8 g) in ethanol (20 ml). The resultant solution was stirred at 25-30° C. temperature for 5 hours. The reaction mixture was cooled to 5-10° C. and stirred for 1.5 to 2 hours. The separated solid was filtered, washed with chilled ethyl acetate (10 ml) and dried under reduced pressure at 50° C. resulting in 20.4 g (98%) of sertraline salicylate (melting point: 161.3-162.4° C.).

### Example 4

### Preparation of Sertraline Adipate

To a solution of sertraline base (14.38 g) in ethyl acetate (300 ml) was added adipic acid (7.2 g). The resultant solution was stirred at 25-30° C. temperature for 5 hours. The solvent was distilled off under reduced pressure and the residue obtained triturated with petroleum ether (80 ml). The slurry was filtered, washed with petroleum ether (10 ml) and dried under reduced pressure at 50° C. resulting in 17.3 g (86%) of sertraline adipate (melting point: 124.3-125.8° C.).

### Example 5

### Preparation of Sertraline 4-methyl benzoate

To a solution of sertraline base (14.38 g) in ethyl acetate (300 ml) was added a solution of 4-methyl benzoic acid (6.4 g) in ethanol (15 ml). The resultant solution was stirred at 25-30° C. temperature for 5 hours. The separated solid was filtered, washed with chilled ethyl acetate (10 ml) and dried under reduced pressure at 50° C. resulting in 15 g (76%) of sertraline 4-methyl benzoate (melting point: 174.7-176.2° C.).

### Example 6

### Preparation of Sertraline 4-methoxy benzoate

To a solution of sertraline base (14.38 g) in ethyl acetate (300 ml) was added a solution of 4-methoxy benzoic acid (7.14 g) in ethanol (15 ml). The resultant solution was stirred at 25-30° C. temperature for 5 hours. The separated solid was filtered, washed with chilled ethyl acetate (10 ml) and dried under reduced pressure at 50° C. resulting in 16 g (78%) of sertraline 4-methoxy benzoate (melting point: 176.4-177.6° C.).

### Example 7

### Preparation of Sertraline Hydrochloride Form II from Sertraline Phthalate

To a mixture of sertraline phthalate (10.0 g) prepared according to example 1 and isobutyl methyl ketone (100 ml), an ethereal solution of HCl (1.0 mole equivalent) was added at 25-35° C. The reaction mass was heated to 70° C. for 3 hours and then cooled to 15-25° C., filtered and washed with isobutyl methyl ketone (20 ml). The obtained solid was dried under reduced pressure at 55° C. resulting in 5.7 g (79%) of sertraline hydrochloride Form-II.

### Example 8

### Preparation of Sertraline Hydrochloride Form II from Sertraline Salicylate

To a mixture of sertraline salicylate (2.0 g) prepared according to example 3 and nitromethane (40 ml), an ethereal solution of HCl (1.05 mole equivalent) was added at 25-35° C. and stirred for 1 hour. The reaction mass was filtered and was washed with nitromethane (10 ml). The obtained solid was dried under reduced pressure at 50° C. resulting in 1.1 g (73%) of sertraline hydrochloride Form-II.

### Example 9

### Preparation of Sertraline Hydrochloride Form II from Sertraline Adipate

To a mixture of sertraline adipate (2.0 grams) prepared according to exaple 4 and nitromethane (40 ml), an ethereal solution of HCl (1.05 mole equivalent) was added at 25-35° C. and stirred for 1 hour. The reaction mass was filtered and was washed with nitromethane (10 ml). The obtained solid was dried under reduced pressure at 50° C. resulting in 1.3 g (86%) of sertraline Hydrochloride Form-II.

### Example 10

### Preparation of Sertraline Hydrochloride Form II from Sertraline 4-methyl benzoate

To a mixture of sertraline 4-methyl benzoate (2.0 g) prepared according to example 5 and nitromethane (40 ml), an ethereal solution of HCl (1.05 mole equivalent) was added at 25-35° C. and stirred for 1 hour. The reaction mass was filtered and was washed with nitromethane (10 ml). The obtained solid was dried under reduced pressure at 50° C. resulting in 1.22 g (79%) of sertraline Hydrochloride Form-II.

### Example 11

### Preparation of Sertraline Hydrochloride Form II from Sertraline 4-methoxy benzoate

To a mixture of sertraline 4-methoxy benzoate (2.0 g) prepared according to example 6 and nitromethane (40 ml), an ethereal solution of HCl (1.05 mole equivalent) was added at 25-35° C. and stirred for 1 hour. The reaction mass was filtered and was washed with nitromethane (10 ml). The obtained solid was dried under reduced pressure at 50° C. resulting in 1.45 g (97%) of Sertraline Hydrochloride Form-II.

### Example 12

### Preparation of Sertraline Hydrochloride Form II from Sertraline Mandalate

To a mixture of sertraline mandelate (10.0 g) and ethyl methyl ketone (100 ml) an ethereal solution of HCl (1.0 mole equivalent) was added at 25-30° C. and stirred at 70° C. for 3 hours. The reaction mass was filtered and was washed with ethyl methyl ketone (20 ml). The obtained solid was dried under reduced pressure at 55° C. resulting in 6.0 g (81%) of Sertraline Hydrochloride Form-II.

### Example 13

### Preparation of Sertraline Hydrochloride Form II from Sertraline Mandalate

10.0 g of sertraline mandelate were suspended in 100 ml of methoxypropyl acetate. A solution of 1.05 molar equivalents of gaseous HCL (approximately 9.5% w/w) in 8.8 g of methoxypropyl acetate was added to the slurry. The slurry was stirred for 1 hour at room temperature and then heated at 70° C. for 2 hours. The product obtained was isolated by filtration, washed with 10 ml of methoxypropyl acetate and dried under reduced pressure at 50-55° C. for 5 hours resulting in 7.1 g (95%) of Sertraline Hydrochloride Form-II.

### Example 14

### Preparation of Sertraline Hydrochloride Form II from Crude Sertraline Hydrochloride

A mixture of crude sertraline hydrochloride (10.0 grams) and nitroethane (200 ml) were heated to 45-50° C. for 1 hour. The reaction mixture was cooled to 25-30° C. and filtered. The solid was washed with nitroethane (40 ml) and dried under reduced pressure at 55° C. The dried solid obtained is sertraline hydrochloride Form-II (9.6 g yield 96%).

### Example 15

### Preparation of Sertraline Hydrochloride Form II from Crude Sertraline Hydrochloride

A mixture of crude sertraline hydrochloride (10.0 grams) and methoxypropyl acetate (200 ml) were heated to 70-75° C. for 3 hours. The reaction mixture was cooled to 25-30° C. and filtered. The solid was washed with methoxypropyl acetate (40 ml) and dried under reduced pressure at 55° C. The dried solid obtained is sertraline hydrochloride Form-II (9.6 g: yield 96%).

### Example 16

### Preparation of Sertraline Hydrochloride Form II from Crude Sertraline Hydrochloride

A mixture of crude sertraline hydrochloride (10.0 grams) and 2-ethoxy ethanol (60 ml) were heated to 60-65° C. for 30 minutes. The reaction mixture was cooled to 25-30° C. and filtered. The solid was washed with 2-ethoxy ethanol (10 ml) and dried under reduced pressure at 55° C. The dried solid obtained is sertraline hydrochloride Form-II (7.2 grams, yield 72%).

1. An acid addition salt of sertraline wherein the acid is selected from phthalic acid, glutamic acid, adipic acid, salicylic acid, 4-methyl benzoic acid and 4-methoxy benzoic acid.

2. A process for preparing an acid addition salt of sertraline according to claim 1, which comprises: (a) mixing sertraline base with an acid selected from phthalic acid, glutamic acid, adipic acid, salicylic acid, 4-methyl benzoic acid and 4-methoxy benzoic acid in an organic solvent, (b) subjecting the mixture for stirring at a suitable temperature and (c) isolating the sertraline salt from the reaction solution by conventional method

3. A process according to claim 2, wherein the organic solvent is selected from methanol, ethanol and/or ethyl acetate.

4. A process for preparing sertraline hydrochloride Form II which comprises (a) slurrying a sertraline acid salt wherein the acid is selected from phthalic acid, glutamic acid, adipic acid, salicylic acid, 4-methyl benzoic acid, 4-methoxy benzoic acid and (R)-mandelic acid in an organic solvent or organic solvent mixture at a temperature between ambient temperature and the boiling point of the solvent or solvent mixture, preferably between 25 and 80° C. (b) treating the slurry with a source of hydrochloric acid and (c) stirring the mixture obtained between ambient temperature and the boiling point of the solvent or solvent mixture for a period of time sufficient to effect the transformation to sertraline hydrochloride Form II.

5. A process according to claim 4, wherein the organic solvent of step (a) is selected from ethyl methyl ketone, acetonitrile, methyl isobutyl ketone, nitromethane, nitroethane, methoxypropyl acetate, 2-ethoxy ethanol, methyl formate, ethylformate, isopropyl acetate, 2-ethoxy ethanol, ethylacetate, n-propylacetate, n-butylacetate and/or isobutyl acetate.

6. A process according to claim 4, wherein the source of hydrochloric acid of step (b) includes gaseous hydrogen chloride, aqueous hydrogen chloride, hydrogen chloride as a solution with an organic solvent or hydrochloride of an organic amide.

7. A pharmaceutical composition comprising crystalline form II of sertraline hydrochloride prepared by using an acid addition salt of sertraline according to claim 1 with pharmaceutically acceptable carriers or excipients.
